SEQUENCE LISTING (8 sequences)

SEQ ID NO: 1 - protein, 213 aa, Homo sapiens
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 5 aa, Homo sapiens
SYNMH

SEQ ID NO: 4 - protein, 11 aa, Homo sapiens
RASSSVSYIHW

SEQ ID NO: 5 - protein, 16 aa, Homo sapiens
AIYPGNGDTSYNQKFK

SEQ ID NO: 6 - protein, 7 aa, Homo sapiens
TSNLASG

SEQ ID NO: 7 - protein, 8 aa, Homo sapiens
YCARSTYY

SEQ ID NO: 8 - protein, 9 aa, Homo sapiens
QWTSNPPTF

(19) United States
(12) Patent Application Publication
(10) Pub. No.: US 2022/0213210 A1
(43) Pub. Date: Jul. 7, 2022

(54) COMBINATION THERAPY OF MULTIPLE SCLEROSIS COMPRISING A CD20 LIGAND

(71) Applicant: MABION SA, Konstantynow Lodzki (PL)

(72) Inventors: Maciej WIECZOREK, Lomianki (PL); Slawomir JAROS, Lodz (PL); Dorota JAROS, Lodz (PL); Julita BALCEREK, Lodz (PL)

(21) Appl. No.: 16/769,790

(22) PCT Filed: Dec. 5, 2018
(86) PCT No.: PCT/EP2018/083597

§ 371 Date: Jun. 4, 2020

(30) Foreign Application Priority Data
Dec. 5, 2017 (EP) 17461640.9

(51) Int. Cl. C07K 16/28; A61K 45/06; A61K 31/277; A61K 31/225; A61K 31/137

(52) U.S. Cl. CPC C07K 16/2887; A61K 45/06; A61K 2039/505; A61K 31/225; A61K 31/137; A61K 31/277

(57) ABSTRACT

The present invention relates to the use of a CD20 ligand and at least one further active agent in the treatment of multiple sclerosis, to the use of a pharmaceutical composition and to a kit comprising the CD20 ligand and at least one further active agent or a pharmaceutical composition comprising such CD20 ligand and further active ingredient.

The present invention relates to the use of a CD20 ligand and at least one further active agent in the treatment of multiple sclerosis, to the use of a pharmaceutical composition and to a kit comprising the CD20 ligand and at least one further active agent or a pharmaceutical composition comprising such CD20 ligand and further active ingredient.

### BACKGROUND OF THE INVENTION

Multiple sclerosis (MS) is a disease of central nervous system and one of the most frequent causes of neurological disabilities. The disease has autoimmunology background and is considered as T-cell mediated.

MS has different clinical phenotypes. The most frequent is the relapsing remitting (RRMS) form characterized by development of acute symptoms of relapses or relapses of neurological deficits followed by a complete or incomplete recovery. Rate of relapses and appearance of new MS lesions are the main factors to be taken under analysis for evaluation of efficacy of anti-inflamatory drugs used for MS treatment, but also there are other factors such as disability progression. After some time most patients develop a phenotype called secondary progressive multiple sclerosis (SPMS). It is characterized by progression of disability which is associated or not with superimposed relapses. The third type of MS is primary progressive multiple sclerosis (PPMS) in which there is slow progression of neurological disability and symptoms without relapses. The fourth type is called progressive-relapsing multiple sclerosis (PRMS). It is characterized by insidious disability progression from onset with some rare superimposed relapses (Castillo-Thivino T. et al., 2013, PLOS ONE, vol. 8, issue 7).

B cells and humoral immunity are also considered as factors involved in MS progression. Such theory is supported by presence of antibodies and complement within active MS lesions, ectopic lymphoid follicles and B-cell related chemokines in the CNS and intrathecally produced immunoglobins (NaismithR. T. et al., 2010, Neurology 74, 1860-1867).

There are several ways of treatment based on small molecules as well as on biologics. Glatiramer acetate is a mix of synthetic peptides with an average length of 40-100 residues. Their sequences resemble myelin basic protein. The mechanisms of action have not been fully understood but probably is related to anti-inflammatory effects by promoting Th2 deviation under the development of Th2 glatiramer acetate reactive CD4+ T cells. These can accumulate in the central nervous system (CNS) and promote bystander suppression by releasing anti-inflammatory cytokines. Glatiramer acetate is administered as subcutaneous injections of 20 mg once a day. Glatiramer acetate is not used for the treatment of SPMS and PPMS forms. Glatiramer acetate treatment is associated with injection-site reactions, such as pain, erythema, swelling and pruritus, transient self-limited systemic reaction of facial flushing and chest tightness, accompanied at times by palpitation, anxiety and dyspnoea. Other reported side effects are lymphadenopathy, dyspnoea and lipoatrophy as permanent and perhaps the most severe side effect (Ø. Torkildsen et al., 2015, European Journal of Neurology, 23 (Suppl. 1): 18-27).

Interferon beta, recombinant forms of polypeptide which naturally is produced by fibroblasts. Probably, the mechanism of action is based on the inhibition of T-lymphocyte proliferation, a shift of cytokine response from an inflammatory response to an anti-inflammatory profile, and reduced migration of inflammatory cells across the blood-brain barrier. Typical dosing of interferon beta-1b is 250 μg subcutaneously every other day; interferon beta-1a is given as a dose of 30 μg intramuscularly once weekly or subcutaneously at doses of 22 or 44 μg three times a week.

Treatment with interferon beta preparations is associated with flu-like symptoms, including muscle aches, fever, chills, headache and back pain (usually 2-8 h after an injection, symptoms resolve within 24 h), elevation of liver enzymes and depression of bone marrow function. Further, cases of severe injection-site reactions involving infection or necrosis as well as severe cases of acute liver failure and pancreatitis have been reported. Long-time exposure to interferon beta does not seem to increase the risk of cancer or infections. As a biologic treatment, interferon beta treatment may induce formation of specific neutralizing antibodies (NABs). The NABs usually are detected between 6 and 18 months of therapy. There is evidence that the efficacy of treatment is reduced in the presence of NABs (Ø. Torkildsen et al., 2015, European Journal of Neurology, 23 (Suppl. 1): 18-27).

Teriflunomide is an immunomodulatory agent that selectively and reversibly inhibits the mitochondrial enzyme dihydroorotate dehydrogenase, required for de novo pyrimidine synthesis. This leads to reduced proliferation of dividing cells that need de novo synthesis of pyrimidine to expand. The therapeutic effect in MS is probably mediated by a reduced number of circulating lymphocytes (Papadopoulou A. et. Al., 2012, Expert Rev Clin Pharmacol, 5: 617-628). It is hypothesized that teriflunomide blocks the proliferation of stimulated B and T lymphocytes, which require de novo synthesis of pyrimidine for their expansion and survival. This is supported by the fact that the anti-proliferative effect of teriflunomide can be overcome in vitro by the addition of exogenous uridine (Ruckemann et al., 1998). The effects of teriflunomide probably are limited to stimulated B and T lymphocytes, as resting lymphocytes and other immune cell lineages can meet their pyrimidine needs from the salvage pathway (Gold and Wolinsky, 2011; Claussen and Kom, 2012). Li Li et. Al (2013, Journal of Neuroimmunology 265, 82-90) investigated the effect of teriflunomide on the activation, proliferation and survival of specific lymphocyte subsets from stimulated peripheral blood mononuclear cells (PBMCs) isolated from healthy human subjects. Additionally, cytokine/chemokine release by stimulated PBMCs were assessed in the presence and absence of teriflunoniide.

Dosing of teriflunomide is 14 mg per day. Treatment with teriflunomide is associated with upper respiratory tract infection, urinary tract infection, paresthesia, diarrhoea, nausea, hair thinning, alanine aminotransferase increase, reduction in blood leucocytes and increase in blood pressure. It can have negative influence on the liver, so alanine aminotransferase screening during the first 6 months of treatment is recommended and thereafter every second month. It is recommended to stop the treatment if liver transaminase levels increase three times above upper normal levels. It is also recommended to do regular monitoring of blood pressure, white blood cells and platelet counts (Ø. Torkildsen et al., 2015, European Journal of Neurology, 23 (Suppl. 1): 18-27).

Dimethyl fumarate is an immunomodulatory agent with anti-inflammatory properties. Dimethyl fumarate is administered twice daily at a dose of 240 mg. The treatment with dimethyl fumarate is associated with flushing, nausea, diarrhea, abdominal pain; upper respiratory tract infection, erythema, injection-site pain, injection-site erythema (Fox R. J. et al., The New England Journal of Medicine, 2012, vol. 367, no 12, 1087-1097), reduction of white blood cell counts and elevations of hepatic transaminases. Similarly to teriflunomide, regular blood tests are recommended. The treatment should be stopped if liver transaminase levels increase three times above upper normal levels.

Fingolimod is an oral sphingosine 1-phosphate receptor (S1PR) modulator that subsequent to its phosphorylation binds with high affinity to S1PR, which in turn leads to an internalization and degradation of the receptor in different tissues and cell types, including lymphocytes. As a consequence, fingolimod inhibits the ability of autoreactive lymphocytes to egress from the lymph nodes towards the CNS. Fingolimod is usually administered 0.5 mg once daily. Treatment with fingolimod is associated with upper respiratory tract infection, headache, cough, diarrhea, back pain, transient bradycardia and atrioventricular block (Ø. Torkildsen et al., 2015, European Journal of Neurology, 23 (Suppl. 1): 18-27). Similarly to dimethyl fumarate and teriflunomide, it can have negative influence on the liver.

Ocrelizumab is a recombinant, humanized, IgG1 class monoclonal antibody targetting CD20 receptor. The CD20 molecule is an activated glycosylated phosphoprotein expressed on a broad range of cells: pre-B cells, naive B cells, memory B cells but it is not expressed on stem cells, pro-B cells, differentiated plasma cells, so the CD20 based treatment does not change concentration of IgG and IgM antibodies in the blood or in the CSF. At least three mechanisms of action are suggested: CDC (complement dependent cytotoxicity), ADCC (antibody dependent cellular cytotoxicity) and apoptosis. Treatment with ocrelizumab is associated with infusion-related reactions, nasopharyngitis, upper respiratory tract infections, headache, and urinary tract infections; in relapsing MS serious adverse events, such as serious infections, and malignancies were reported (Kappos L., 2017, Neurology Reviews, July: 25(7):1, 22).

Rituximab is a recombinant, chimeric, IgG1 class antibody. Similarly to ocrelizumab, it targets CD20 receptor on B lymphocytes. Mechanisms of action are considered to be the same as for ocrelizumab. Data regarding the safety profile of rituximab in MS patients is limited, due the fact that it comes from relatively short time of treatment and limited population. There were observed frequent but not serious infusion-related reactions which usually are reduced with subsequent infusions (Castillo-Trivino T. et. al., 2013, PLOS ONE, vol. 8, issue 7).

Suming up, there are already available treatments for MS. However, most of the available drugs have an unfavorable safety profile. All the mentioned therapies have numbers of adverse reactions described. In case of chemical compounds, one of the major issues is a negative influence on the liver and the need for regular monitoring leading to termination of treatment. Thus, there remains a need for MS therapies increasing safety without decrease of efficacy.

### SUMMARY OF THE INVENTION

In a first aspect, the present invention provides a CD20 ligand and at least one further active agent for use in the treatment of multiple sclerosis, wherein the at least one further active agent decreases lymphocyte count.

In a second aspect, the present invention relates to a pharmaceutical composition for use in the treatment of multiple sclerosis comprising the CD20 ligand and the at least one further active agent according to the first aspect of the invention and at least one pharmaceutically acceptable excipient.

In a third aspect, the present invention relates to a kit for use in the treatment of multiple sclerosis comprising the CD20 ligand and the at least one further active agent according to first aspect of the invention or a composition according to the second aspect of the invention.

### LIST OF FIGURES

In the following, the content of the figures comprised in this specification is described. In this context please also refer to the detailed description of the invention above and/or below.

FIG. 1: Combination of MabionCD20DP and teriflunomide. MabionCD20DP and teriflunomide have been administered to an experimental allergic encephalomyelitis (EAE) mouse model. The MOG (myelin oligodendrocyte glycoprotein)-induced EAE murine model consists of a sensitization period, induced by the single SC injection of MOG emulsified in CFA (complete Freund's adjuvant) on Day 0, followed by IP supplemental immunostimulation with PT carried out once at the time of EAE induction and once again 48 hours later. FIG. 1 shows the results of the treatment of MabionCD20DP and teriflunomide.

FIG. 2: Combination of MabionCD20DP and dimethyl fumarate. MabionCD20DP and dimethyl fumarate have been administered to an experimental allergic encephalomyelitis (EAE) mouse model. The MOG-induced EAE murine model consists of a sensitization period, induced by the single SC injection of MOG emulsified in CFA on Day 0, followed by IP supplemental immnunostimulation with PT carried out once at the time of EAE induction and once again 48 hours later. FIG. 2 shows the results of the treatment of MabionCD20DP and dimethyl fumarate.

FIG. 3: Combination of MabionCD20DP and fingolimod. MabionCD20DP and fingolimod have been administered to an experimental allergic encephalomy elitis (EAE) mouse model. The MOG-induced EAE murine model consists of a sensitization period, induced by the single SC injection of MOG emulsified in CFA on Day 0, followed by IP supplemental immunostimulation with PT carried out once at the time of EAE induction and once again 48 hours later. FIG. 3 shows the results of the treatment of MabionCD20DP and fingolimod.

### DETAILED DESCRIPTIONS OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

As used herein, “CD20” is an activated-glycosylated phosphoprotein expressed on the surface of all B-cells. The sequence of human CD20 is indicted in GenBank reference NP_690605.

The term “ligand” as used herein includes but is not limited to antigen binding protein antibodies and fragments thereof.

The term “antigen binding protein” as used herein, refers to immunoglobulin molecules and immunologically active portions of immunoglobulin molecules, i.e. molecules that contain an antigen-binding site that immunospecifically binds an antigen. Also comprised are immunoglobulin-like proteins that are selected through techniques including, for example, phage display to specifically bind to a target molecule or target epitope. In assessing the binding and/or specificity of an antigen binding protein, e.g., an antibody or immunologically functional fragment thereof, an antibody or fragment can substantially inhibit binding of a ligand to its binding partner when an excess of antibody reduces the quantity of binding partner bound to the ligand by at least about 1-20%, 20-30%, 30-40%, 40-50%, 50-60%, 60-70%, 70-80%, 80-85%, 85-90%, 90-95%, 95-97%, 97-98%, 98-99% or more (e.g. as measured in an in vitro competitive binding assay). The neutralizing ability may be described in terms of an IC₅₀ or EC₅₀ value.

The “IC₅₀” value refers to the half maximal inhibitory concentration of a substance and is thus a measure of the effectiveness of a substance in inhibiting a specific biological or biochemical function. The values are typically expressed as molar concentration. The IC₅₀ of a drug can be determined in functional antagonistic assays by constructing a dose-response curve and examining the inhibitory effect of the examined substance at different concentrations. Alternatively, competition binding assays may be performed in order to determine the IC₅₀ value. Typically, inhibitory antibodies of the present invention exhibit an IC₅₀ value of between 50 nM-1 pM, more preferably 10 nM to 10 pM, and even more preferably between 1 nM and 50 pM, i.e. 50 nM, 10 nM, 1 nM, 900 pM, 800 pM, 700 pM, 600 pM, 500 pM, 400 pM, 300 pM, 200 pM, 0 to pM, 50 pM, or 1 pM.

The “ECs,” value refers to half maximal effective concentration of a substance and is thus a measure of the concentration of said substance which induces a response halfway between the baseline and maximum after a specified exposure time. It is commonly used as a measure of drug's potency. The EC₅₀ of a graded dose response curve therefore represents the concentration of a substance where 50% of its maximal effect is observed. The EC₅₀ of a quantal dose response curve represents the concentration of a compound where 50% of the population exhibit a response, after a specified exposure duration. Typically, inhibitory antibodies of the present invention exhibit an EC₅₀ value of between 50 nM to 1 pM, more preferably 10 nM to 10 pM, and even more preferably between 1 nM and 50 pM, i.e. 50 nM, 10 nM, 1 nM, 900 pM, 800 pM, 700 pM, 600 pM, 500 pM, 400 pM, 300 pM, 200 pM, 100 pM, 50 pM, or 1 pM.

The term “antibody” typically refers to a glycoprotein comprising at least two heavy (H) chains and two light (L) chains inter-connected by disulfide bonds, or an antigen-binding portion thereof. The term “antibody” also includes all recombinant forms of antibodies, in particular of the antibodies described herein, e.g. antibodies expressed in prokaryotes, unglycosylated antibodies, and any antigen-binding antibody fragments and derivatives as described below. Each heavy chain is comprised of a heavy chain variable region (abbreviated herein as VH or V_{H}) and a heavy chain constant region. Each light chain is comprised of a light chain variable region (abbreviated herein as VL or V_{L}) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody (or simply “fragment”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) Fab fragments, monovalent fragments consisting of the VL, VH, CL and CH domains; (ii) F(ab′)₂ fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al. (1989) Nature 341: 544-546), which consist of a VH domain; (vi)isolated complementarity determining regions (CDR), and (vii) combinations of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding fragment” of an antibody. A further example is a binding-domain immunoglobulin fusion protein comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. The binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies. Further examples of “antigen-binding fragments” are so-called microantibodies, which are derived from single CDRs. For example, Heap et al., 2005, describe a 17 amino acid residue microantibody derived from the heavy chain CDR3 of an antibody directed against the gp120 envelope glycoprotein of HIV-1. Other examples include small antibody mimetics comprising two or more CDR regions that are fused to each other, preferably by cognate framework regions. Such a small antibody mimetic comprising V_{H} CDR1 and V_{L} CDR3 linked by the cognate V_{H} FR2 has been described by Qiu et al., 2007.

Thus, the term “antibody or antigen-binding fragment thereof”, as used herein, refers to immunoglobulin molecules and immunologically active portions of immunoglobulin molecules, i.e. molecules that contain an antigen-binding site that immunospecifically binds an antigen. Also comprised are immunoglobulin-like proteins that are selected through techniques including, for example, phage display to specifically bind to a target molecule or target epitope, e.g. to CD20. The immunoglobulin molecules of the invention can be of any type (e.g., IgG, IgE, IgM IgD, IgA and IgY), class (e.g., IgG1, IgG2, preferably IgG2a and IgG2b, IgG3, IgG4, IgA1 and IgA2) or subclass of immunoglobulin molecule.

Antibodies and antigen-binding fragments thereof usable in the invention may be from any animal origin including birds and mammals. Preferably, the antibodies or fragments are from human, chimpanzee, rodent (e.g. mouse, rat, guinea pig, or rabbit), chicken, turkey, pig, sheep, goat, camel, cow, horse, donkey, cat, or dog origin. It is particularly preferred that the antibodies are of human or murine origin. Antibodies of the invention also include chimeric molecules in which an antibody constant region derived from one species, preferably human, is combined with the antigen binding site derived from another species, e.g. mouse. Moreover antibodies of the invention include humanized molecules in which the antigen binding sites of an antibody derived from a non-human species (e.g. from mouse) are combined with constant and framework regions of human origin.

As used herein, “human antibodies” include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). Hunan antibodies of the invention include antibodies isolated from human immunoglobulin libraries or from animals transgenic for one or more human immunoglobulin and that do not express endogenous immunoglobulins, as described for example in U.S. Pat. No. 5,939,598 by Kucherlapati & Jakobovits.

The term “monoclonal antibody” as used herein refers to a preparation of antibody molecules of single molecular composition. A monoclonal antibody displays a single binding specificity and affinity for a particular epitope. In one embodiment, the monoclonal antibodies are produced by a hybridoma which includes a B cell obtained from a non-human animal, e.g. mouse, fused to an immortalized cell.

The term “recombinant antibody”, as used herein, includes all antibodies that are prepared, expressed, created or isolated by recombinant means, such as (a) antibodies isolated from an animal (e.g., a mouse) that is transgenic or transchromosomal with respect to the immunoglobulin genes or a hybridoma prepared therefrom, (b) antibodies isolated from a host cell transformed to express the antibody, e.g. from a transfectoma, (c) antibodies isolated from a recombinant, combinatorial antibody library, and (d) antibodies prepared, expressed, created or isolated by any other means that involve splicing of immunoglobulin gene sequences to other DNA sequences.

The term “chimeric antibody” refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. One clear advantage to such chimeric forms is that the variable region can conveniently be derived from presently known sources using readily available B-cells or hybridomas from non-human host organisms in combination with constant regions derived from, for example, human cell preparations. While the variable region has the advantage of ease of preparation and the specificity is not affected by the source, the constant region being human is less likely to elicit an immune response from a human subject when the antibodies are injected than would the constant region from a non-human source. However, the definition is not limited to this particular example.

The term “humanized antibody” refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified by one or more amino acid substitutions, e.g. modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

Different methods for humanizing antibodies are known to the skilled person, as reviewed by Almagro & Fransson, 2008, the content of which is herein incorporated by reference in its entirety. The review article by Almagro & Fransson is briefly summarized in the following. Almagro & Fransson distinguish between rational approaches and empirical approaches. Rational approaches are characterized by generating few variants of the engineered antibody and assessing their binding or any other property of interest. If the designed variants do not produce the expected results, a new cycle of design and binding assessment is initiated. Rational approaches include CDR grafting, Resurfacing, Superhumanization, and Human String Content Optimization. In contrast, empirical approaches are based on the generation of large libraries of humanized variants and selection of the best clones using enrichment technologies or high-throughput screening. Accordingly, empirical approaches are dependent on a reliable selection and/or screening system that is able to search through a vast space of antibody variants. In vitro display technologies, such as phage display and ribosome display fulfill these requirements and are well-known to the skilled person. Empirical approaches include FR libraries, Guided selection., Framework-shuffling, and Humaneering.

A “bivalent antibody” comprises two antigen binding sites. Bivalent antibodies may be monospecific or bispecific. In case, the bivalent antibody is monospecific, the two binding sites of the antibody have the same antigen specificities. A “bispecific” or “bifunctional” antigen binding protein or antibody is a hybrid antigen binding protein or antibody, respectively, having two different antigen binding sites. The two binding sites of a bispecific antigen binding protein or antibody bind to two different epitopes residing either on the same or on different antigens. Bispecific antigen binding proteins and antibodies are a species of multispecific antigen binding protein antibody and can be produced by a variety of methods including, but not limited to, fusion of hybridomas, chemical linking of IgG or IgG fragments such as Fab′, or by genetic means. See, e.g., Songsivilai and Lachmann, 1990, Clin. Exp. Immunol. 79:315-321; Kostelny et al., 1992, J. Immunol. 148:1547-1553; Kontermann, 2014, MAbs 4:182-197.

A “trifunctional antibody” is a type of bispecific antibody which comprises the two binding sites targeting different antigens as well as an intact Fe-part which can bind to an Fe receptor on accessory cells (e.g. monocytes/macrophages, natural killer cells, dendritic cells or other). For example, a trifunctional antibody may comprise a binding site targeting an epitope on the surface of a cancer cell, the second binding site may target an epitope on the surface of a T cell (e.g. CD3) and the Fe-part may bind to the Fe receptor on the surface of a macrophage. Such trifunctional antibody is thus able to link T cells and macrophages to the tumor cells, leading to their destruction.

Papain digestion of antibodies produces two identical antigen binding fragments, called “Fab fragments” (also referred to as “Fab portion” or “Fab region”) each with a single antigen binding site, and a “Fc fragment” (also referred to as “Fe portion” or “Fe region”) whose name reflects its ability to crystallize readily. The crystal structure of the human IgG Fe region has been determined (Deisenhofer (1981) Biochemistry 20:2361-2370). In IgG, IgA and IgD isotypes, the Fe region is composed of two identical protein fragments, derived from the CH2 and CH3 domains of the antibody's two heavy chains; in IgM and IgE isotypes, the Fe regions contain three heavy chain constant domains (CH2-4) in each polypeptide chain. In addition, smaller immunoglobulin molecules exist naturally or have been constructed artificially. The term “Fab′ fragment” refers to a Fab fragment additionally comprise the hinge region of an Ig molecule whilst “F(ab′)2 fragments” are understood to comprise two Fab′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al. (1988) Proc. Natl. Acad. Sci. USA 85, 5879-5883). Divalent single-chain variable fragments (di-scFvs) can be engineered by linking two scFvs (scFvA-scFvB). This can be done by producing a single peptide chain with two V_{H} and two V_{L} regions, yielding “tandem scFvs” (V_{H}A-V_{L}A-V_{H}B-V_{L}B). Another possibility is the creation of scFvs with linkers that are too short for the two variable regions to fold together, forcing scFvs to dimerize. Usually linkers with a length of 5 residues are used to generate these dimers. This type is known as “diabodies”. Still shorter linkers (one or two amino acids) between a V-H and VL domain lead to the formation of monospecific trimers, so-called “triabodies” or “tribodies”. Bispecific diabodies are formed by expressing to chains with the arrangement V_{H}A-V_{L}B and V_{H}B-V_{L}A or V_{L}A-V_{H}B and V_{L}B-V_{H}A, respectively. Single-chain diabodies (scDb) comprise a V_{H}A-V_{L}B and a V_{H}B-V_{L}A fragment which are linked by a linker peptide (P) of 12-20 amino acids, preferably 14 amino acids, (V_{H}A-V_{L}B-V_{H}B-V_{L}A). “Bi-specific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

As used herein, the term “antibody-like protein” or immunoglobulin-like protein refers to a protein that has been engineered (e.g. by mutagenesis of loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Antibody-like proteins include without limitation affibodies, anticalins, and designed ankyrin repeat proteins (for review see: Binz H. K. et al. (2005) Engineering novel binding proteins from nonimmunoglobulin domains. Nat. Biotechnol. 23(10):1257-1268). Antibody-like proteins can be derived from large libraries of mutants, e.g. be panned from large phage display libraries and can be isolated in analogy to regular antibodies. Also, antibody-like binding proteins can be obtained by combinatorial mutagenesis of surface-exposed residues in globular proteins. Antibody-like proteins are sometimes referred to as “peptide aptamers”.

Thus, “antibodies and antigen-binding fragments thereof” suitable for use in the present invention include, but are not limited to, polyclonal, monoclonal, monovalent, bispecific, heteroconjugate, multispecific, recombinant, heterologous, heterohybrid, chimeric, humanized (in particular CDR-grafted), deimmunized, or human antibodies, Fab fragments, Fab′ fragments, F(ab′)₂ fragments, fragments produced by a Fab expression library, Fd, Fv, disulfide-linked Fvs (dsFv), single chain antibodies (e.g. scFv), diabodies or tetrabodies (Holliger P. et al. (1993) Proc. Natl. Acad. Sci. U.S.A. 90(14), 6444-6448), nanobodies (also known as single domain antibodies), anti-idiotypic (anti-Id) antibodies (including, e.g., anti-Id antibodies to antibodies of the invention), and epitope-binding fragments of any of the above.

The antibodies described herein are preferably isolated. An “isolated antibody” as used herein, is intended to refer to an antibody which is substantially free of other antibodies having different antigenic specificities (e.g., an isolated antibody that specifically binds to CD20 is substantially free of antibodies that specifically bind antigens other than CD20). An isolated antibody that specifically binds to an epitope, isoform or variant of human CD20 may, however, have cross-reactivity to other related antigens, e.g. from other species (e.g. CD20 species homologs, such as rat CD20). Moreover, an isolated antibody may be substantially free of other cellular material and/or chemicals. In one embodiment of the invention, a combination of “isolated” monoclonal antibodies relates to antibodies having different specificities and being combined in a well-defined composition.

The term “active agent” as used herein, refers to any therapeutic activity an agent may exhibit.

Multiple sclerosis is a demyelinating disease in which the insulating covers of nerve cells in the brain and spinal cord are damaged. The most important phenotypes of MS are clynically isolated syndrome (CIS), relapsing remitting (RRMS), secondary progressive multiple sclerosis (SPMS), primary progressive multiple sclerosis (PPMS) and progressive-relapsing multiple sclerosis (PRMS).

The term “lymphocyte count” as used herein refers to the number of lymphocytes in the blood or other biological samples. Preferably, the lymphocyte count is the number if lymphocytes in the blood.

The term “compete” when used in the context of antigen binding proteins that compete for the same epitope means competition between antigen binding proteins as determined by an assay in which the antigen binding protein (e.g., antibody or immunologically functional fragment thereof) being tested prevents or inhibits (e.g., reduces) specific binding of a reference antigen binding protein (e.g., a ligand, or a reference antibody) to a common antigen (e.g., CD20 or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Usually, when a competing antigen binding protein is present in excess, it will inhibit (e.g., reduce) specific binding of a reference antigen binding protein to CD20 or an extracellular fragment thereof by at least about 40-45%, 45-50%, 50-55%, 55-60% 60-65%, 65-70% or 70-75%, such as about 75% or more. In some instances, binding is inhibited by at least about 80-85%, 85-90%, 90-95% or 95-97%, such as about 97% or more.

The term “binding” according to the invention preferably relates to a specific binding. The term “binding affinity” generally refers to the strength of the sum total of noncovalent interactions between a single binding site of a molecule (e.g., an antibody) and its binding partner (e.g., target or antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). “Specific binding” means that a binding moiety (e.g. an antibody) binds stronger to a target such as an epitope for which it is specific compared to the binding to another target. A binding moiety binds stronger to a first target compared to a second target if it binds to the first target with a dissociation constant (K_{d}) which is lower than the dissociation constant for the second target. The dissociation constant (Ki) for the target to which the binding moiety binds specifically is more than 10-fold, preferably more than 20-fold, more preferably more than 50-fold, even more preferably more than 100-fold, 200-fold, 500-fold or 1000-fold lower than the dissociation constant (K_{d}) for the target to which the binding moiety does not bind specifically.

Accordingly, the term “K_{d}” (measured in “mol/L”, sometimes abbreviated as “M”) is intended to refer to the dissociation equilibrium constant of the particular interaction between a binding moiety (e.g. an antibody or fragment thereof) and a target molecule (e.g. an antigen or epitope thereof). Affinity can be measured by common methods known in the art, including but not limited to surface plasmon resonance based assay (such as the BIAcore assay); quartz crystal microbalance assays (such as Attana assay); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

Typically, ligands according to the invention bind with a sufficient binding affinity to their target, for example, with a K_{d} value of between 500 nM-1 pM, i.e. 500 nM, 450 nM, 400 nM, 350 nM, 300 nM, 250 nM, 200 nM, 150 nM, 100 nM, 50 nM, 10 nM, 1 nM, 900 pM, 800 pM, 700 pM, 600 pM, 500 pM, 400 pM, 300 pM, 200 pM, 100 pM, 50 pM, 1 pM.

The term “small molecule drug” as used herein, refers to a low molecular weight (<900 Daltons) compound. “Pharmaceutically acceptable” means approved by a regulatory agency of the Federal or a state government or listed in the U.S. PharMacopeia, European Phannacopeia (Ph. Eur.) or other generally recognized pharmacopeia for use in animals, and more particularly in humans.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

### EMBODIMENTS

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides a CD20 ligand and at least one further active agent for use in the treatment of multiple sclerosis, wherein the at least one further active agent decreases lymphocyte count. Preferably, the lymphocyte count is decreased by at least 40%, preferably by at least 35%, preferably by at least 30%, more preferably by at least 25%, most preferably by at least 15% relative to baseline value. The percentage of decreased lymphocyte count can be measured by art known methods, including the TruCount test (Becton Dickinson Immunocytometry Systems, San Jose, Calif.), which uses tubes containing a known number of fluorescent polystyrene beads. The absolute number of cells is calculated based on the ratio of beads to cells counted in the tube. The advantages of this test are that it does not rely on hematology determinations for an absolute lymphocyte count, is suitable for specimens older than 18 h, and provides percentages as well as absolute numbers of lymphocytes such as CD4+T cells (Nicholson JK. Stein D. Mui T, Mack R, Hubbard M. Denny T. Evaluation of a method for counting absolute numbers of cells with a flow cytometer. Clin Diagn Lab Immunol 1997; 4: 309-313).

Alternativly one of the methods listed below can be used to determine lymphocyte count:

Methods for Absolute Cell Subset Counting
a) Cytometric Methods

| | |
|---|---|
| 1) | ECM immunophenotyping + Hematology (Dual-Platform Approach) |

a1) Volumetric Technologies

| | |
|---|---|
| 2) | ORTHO Cytoron Absolute + Immunocount II Software + |
| | Trio Reagents |
| 3) | DAKO GALAXY-PARTEC PAS Instrument |
| 4) | BIOMETRIC IMAGN 2000 + Dedicated Cartridges |

a2) Microbead-Based Technologies

| | |
|---|---|
| 5) | BECTON DICKINSON FACSCount + Dedicated Cartridges |
| 6) | BECTON DICKINSON TRUCount Tubes + 3 and 4 Color Reagents |
| 7) | BECKMAN-COULTER Flowcount Beads + 3 and 4 Color Reagents |
| 8) | BANGS-FCSC Count Standard |

b) Hematologic Cytometric-Like Methods

| | |
|---|---|
| 9) | BAYER H2 + Dedicated Module (Absorption Immunocytochemistry) |
| 10) | COULTER STKS-Gen's + VCS Module |
| 11) | ABBOTT Cell-Dyn 4000 |

c) Non-Cytometric Methods

| | |
|---|---|
| 12) | Beckman-Coulter Manual CD4 Count Kit |
| 13) | T Cell Science TraX CD4 Kit |
| 14) | Zynaxis Zymmune CD4/CD8 |
| 15) | Pasteur-Sanofi Capcellia CD4/CD8 |
| 16) | DuPont Medical CD4-CCS Kit |

In a preferred embodiment of the first aspect of the present invention, the CD20 ligand is an antigen binding protein, an antibody or a fragment thereof. In some embodiments, an antigen binding protein specifically binds to the same epitope that is bound by any of the antibodies (the reference antibodies) disclosed herein or competes with such an antibody for binding to CD20.

In a preferred embodiment of the first aspect of the present invention, the CD20 ligand is an antibody or a fragment thereof. In an even more preferred embodiment, the CD20 ligand is an antibody. For example, the CD20 ligand is MabionCD20DP (light chain variable domain: SEQ ID NO: 1; heavy chain variable domain: SEQ ID NO: 2). The antibody according to the invention without limitation can be selected from a human antibody, a humanized antibody, a monoclonal antibody, a recombinant antibody and a chimeric antibody.

In a preferred embodiment of the first aspect of the present invention, the CD20 ligand comprises a combination of a light chain variable domain and a heavy chain variable domain selected from the group of combinations consisting of:

a light chain variable domain having a sequence with at least 90% identity to SEQ ID NO: 1 and

a heavy chain variable domain having a sequence with at least 90% identity to SEQ ID NO: 2.

In a preferred embodiment of the first aspect of the present invention, the CD20 ligand comprises a complementary determining region 3 of the heavy chain (CDRH3) comprising or consisting of the amino acid sequence of SEQ ID NO: 7 and a complementary determining region 3 of the light chain (CDRL3) comprising or consisting of the amino acid sequence of SEQ ID NO: 8. More preferably, the CD20 ligand further comprises one or more selected from the group consisting of a CDRH1 comprising or consisting of the amino acid sequence of SEQ ID NO: 3, a CDRH-2 comprising or consisting of the amino acid sequence of SEQ ID NO: 5, a CDRL1 comprising or consisting of the amino acid sequence of SEQ ID NO: 4 and a CDRL2 comprising or consisting of the amino acid sequence of SEQ ID NO: 6.

In a preferred embodiment of the first aspect of the present invention, the CD20 ligand competes for binding to CD20 with at least one antibody selected from the group consisting of rituximab, ibritumomab, obinutuzumab, ofatumumab, tositumomab and ocrelizumab. Preferably, the CD20 ligand competes for binding to CD20 with rituximab.

In a preferred embodiment of the first aspect of the present invention, the active agent is a small molecule drug.

In a preferred embodiment of the first aspect of the present invention, the active agent is (i) an inhibitor of activated T-cells and/or B-cells; (ii) an antagonist of transcription factor NF-_{κ}B; (iii) an antagonist of sphingosine-1-phosphate (SIP) receptor(s), preferably selected from the group consisting of fingolimod, ponesimod (ACT128800), siponimod (BAF312), ozanimod (RPC1063), ceralifimod (ONO-4641). GSK2018682, and MT-1303, (iv) an activator of the nuclear factor (erythroid-derived 2)-like 2 (Nrf2) pathway; and/or (v) an inhibitor of dihydroorotate dehydrogenase, preferably selected from the group leflunomide and teriflunomide.

In a preferred embodiment of the first aspect of the present invention, the active agent is a compound according to Formula I:

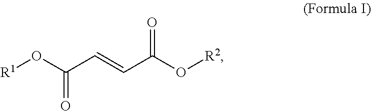

wherein R¹ and R² are the same or different and are independently selected from the group consisting of linear, branched or cyclic, saturated or unsaturated C₁₋₂₀ alkyl which may be optionally substituted with halogen (Cl F, I, or Br), hydroxy, C₁₋₄ alkoxy, nitro or cyano; preferably wherein R¹ and R² are independently selected from the group consisting of C₁₋₅ alkyl, such as methyl, ethyl, n-propyl, isopropyl, n-butyl, sec-butyl, t-butyl, pentyl, cyclopentyl more preferably wherein R¹ and R² are the same; even more preferably, wherein R¹ and R² are methyl; or a pharmaceutically acceptable form thereof, including any pharmaceutically acceptable salts, prodrugs, racemic mixtures, conformational and/or optical isomers, crystalline polymorphs and isotopic variants. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In a preferred embodiment of the first aspect of the present invention, the active agent is a compound according to Formula II:

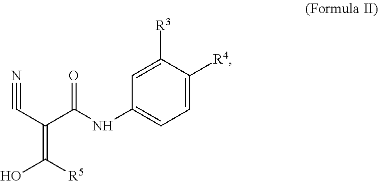

wherein R³ and R⁴ are the same or different and are independently selected from the group consisting of carboxyl, halogen, hydrogen, trihalomethyl and NO₂, substituted or unsubstituted aryl; and R⁵ is selected from the group consisting of aryl, alkyl, alkenyl and alkynyl; preferably, wherein R³ and R⁴ are the same or different and are hydrogen or trihalomethyl and R⁵ is alkyl; or a pharmaceutically acceptable form thereof, including any pharmaceutically acceptable salts, prodrugs, racemic mixtures, conformational and/or optical isomers, crystalline polymorphs and isotopic variants. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In a preferred embodiment of the first aspect of the present invention, the active agent is a compound according to Formula III:

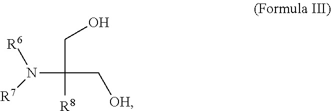

wherein R⁶ and R⁷ are the same or different and are independently selected from the group consisting of hydrogen, alkyl, or acyl; R⁸ is a phenylakyl wherein alkyl is a straight- or branched C₆-C₂₀ carbon chain; or a phenylalkyl wherein alkyl is a straight- or branched C₁-C₃₀ carbon chain wherein said phenylalkyl is substituted by a straight- or branched C₆₋₂₀ carbon chain optionally substituted by halogen, a straight- or branched C₆₋₂₀ alkoxy chain optionally substituted by halogen, a straight- or branched C₆₋₂₀ alkenyloxy, phenyl-C₁₋₁₄alkoxy, halophenyl-C₁₋₄alkoxy, phenyl-C₁₋₁₄alkoxy-C₁₋₁₄alkyl, phenoxy-C₁₋₄alkoxy or phenoxy-C₁₋₄ alkyl, cycloalkylalkyl substituted by C₆₋₂₀alkyl, heteroarylalkyl substituted by C₆₋₂₀alkyl, heterocyclic C₆₋₂₀alkyl or heterocyclic alkyl substituted by C₂₋₂₀alkyl; preferably wherein R⁶ and R⁷ are the same and are selected from the group consisting of hydrogen and alkyl and wherein R⁸ is optionally substituted 2-(4-octylphenyl)ethyl; or a pharmaceutically acceptable form thereof, including any pharmaceutically acceptable salts, prodrugs, racemic mixtures, conformational and/or optical isomers, crystalline polymorphs and isotopic variants. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In a preferred embodiment of the first aspect of the present invention, the active agent is selected from the group consisting of dimethyl fumarate, leflunomide, teriflunomide and fingolimod or a pharmaceutically acceptable form thereof, including any pharmaceutically acceptable salts, prodrugs, racemic mixtures, conformational and/or optical isomers, crystalline polymorphs and isotopic variants. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. In one embodiment, the CD20 ligand, preferably MabionCD20DP, is administered together with dimethyl fumarate or a pharmaceutically acceptable form thereof. In one embodiment, the CD20 ligand, preferably MabionCD20DP, is administered together with leflunomide or a pharmaceutically acceptable form thereof. In one embodiment, the CD20 ligand, preferably MabionCD20DP, is administered together with teriflunomide or a pharmaceutically acceptable form thereof. In one embodiment, the CD20 ligand, preferably MabionCD20DP, is administered together with fingolimod or a pharmaceutically acceptable form thereof.

In a preferred embodiment of the first aspect of the present invention, multiple sclerosis is selected from the group consisting of clinically isolated syndrome (CIS), relapsing-remitting multiple sclerosis (RRMS), primary progressive multiple sclerosis (PPMS), progressive-relapsing multiple sclerosis (PRMS) and secondary progressive multiple sclerosis (SPMS). Relapsing-remitting MS (RRMS) is characterized by unpredictable relapses followed by remission (from months to years). Complete resolution may occur, but mild residual symptoms or signs persist in up to 40% of attacks. This describes the initial course of 80% of individuals with MS. When deficits always resolve between attacks, this is sometimes referred to as benign MS, although people will still build up some degree of disability in the long term. On the other hand, the term malignant multiple sclerosis is used to describe people with MS having reached significant level of disability in a short period. The relapsing-remitting subtype usually begins with a clinically isolated syndrome (CIS). In CIS, a person has an attack suggestive of demyelination, but does not fulfill the criteria for multiple sclerosis. 30 to 70% of persons experiencing CIS later develop MS.

Primary progressive MS occurs in approximately 10-20% of individuals, with no remission after the initial symptoms. It is characterized by progression of disability from onset, with no, or only occasional and minor, remissions and improvements. The usual age of onset for the primary progressive subtype is later than of the relapsing-remitting subtype. It is similar to the age that secondary progressive usually begins in relapsing-remitting MS, around 40 years of age.

Progressive-relapsing multiple sclerosis (PRMS) is characterized by steadily worsening neurologic function from the beginning with occasional relapses. Patients with progressive-relapsing multiple sclerosis (PRMS) may experience increasing level of disability by incomplete recovery from acute exacerbations and by ongoing deterioration. In progressive-relapsing multiple sclerosis (PRMS), disability accumulates solely by continuous decline. It is considered the least common form of MS, but relapses are reportedly uncommon (Tullman MJ, 2004, Mult Scler., August; 10(4):451-4).

Secondary progressive MS occurs in around 65% of those with initial relapsing-remitting MS, who eventually have progressive neurologic decline between acute attacks without any definite periods of remission. Occasional relapses and minor remissions may appear. The most common length of time between disease onset and conversion from relapsing-remitting to secondary progressive MS is 19 years.

In a preferred embodiment of the first aspect of the present invention, the CD20 ligand and/or the at least one further active agent are administered in a sub-therapeutic dose. A sub-therapeutic dose as used herein, refers to a dose that is less than about 75%, less than about 70%, less than about 60%, less than about 50%, less than about 40%, less than about 30%, less than about 25%, less than about 20%, less than about 15%, less than about 10%, or less than about 5% of the dose that is conventionally administered as a therapeutic dose.

In one embodiment, only the CD20 ligand is administered in a sub-therapeutic dose, i.e. in a dose that is less than about 75%, less than about 70%, less than about 60%, less than about 50%, less than about 40%, less than about 30%, less than about 25%, less than about 20%, less than about 15%, less than about 10%, or less than about 5% of the dose that is conventionally administered of the CD20 ligand. For example, if the CD20 ligand is MabionCD20DP, MabionCD20DP is administered in a sub-therapeutic dose, i.e. in a dose that is less than about 75%, less than about 70%, less than about 60%, less than about 50%, less than about 40%, less than about 30%, less than about 25%, less than about 20%, less than about 15%, less than about 10%, or less than about 5% of the dose that is conventionally administered of MabionCD20DP in the treatment of MS. For example, a typical dose of MabionCD20DP or rituximab in the treatment of MS is 375 mg/m² body surface. Accordingly, a sub-therapeutic dose of MabionCD20DP or rituximab is a dose that is less than 280 mg/m² body surface, less than 260 mg/m² body surface, less than 185 mg/m² body surface, less than 150 mg/m² body surface, less than 110 mg/m² body surface, less than 95 mg/m body surface, less than 75 mg/m² body surface, less than 55 mg/m² body surface, less than 35 mg/m² body surface or less than 18 mg/m² body surface.

In another embodiment, only the at least one further active agent is administered in a sub-therapeutic dose, i.e. in a dose that is less than about 75%, less than about 70%, less than about 60%, less than about 50%, less than about 40%, less than about 30%, less than about 25%, less than about 20%, less than about 15%, less than about 10%, or less than about 5% of the dose that is conventionally administered of the at least one further active agent. For example, if the at least one further active agent is a compound of formula I, preferably teriflunomide, the compound of formula T, preferably teriflunomide is administered in a sub-therapeutic dose, i.e. in a dose that is less than about 75%, less than about 70%, less than about 60%, less than about 50%, less than about 40%, less than about 30%, less than about 25%, less than about 20%, less than about 15%, less than about 10%, or less than about 5% of the dose that is conventionally administered of teriflunomide. The conventionally administered dose of teriflunomide is 14 mg. Accordingly, a sub-therapeutic dose of teriflunomide is less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

If the at least one further active agent is a compound of formula II, preferably fingolimod, the compound of formula II, preferably fingolimod is administered in a sub-therapeutic dose, i.e. in a dose that is less than about 75%, less than about 70%, less than about 60%, less than about 50%, less than about 40%, less than about 30%, less than about 25%, less than about 20%, less than about 15%, less than about 10%, or less than about 5% of the dose that is conventionally administered of fingolimod. The conventially administered dose of fingolimod is 500 μg. Accordingly, a sub-therapeutic dose of fingolimod is less than 375 μg, less than 350 μg, less than 300 μg, less than 250 μg, less than 200 μg, less than 150 μg, less than 125 μg, less than 100 μg, less than 75 μg, less than 50 μg or less than 25 μg.

If the at least one further active agent is a compound of formula III, preferably dimethyl fumarate, dimethyl fumarate is administered in a sub-therapeutic dose, i.e. in a dose that is less than about 75%, less than about 70%, less than about 60%, less than about 50%, less than about 40%, less than about 30%, less than about 25%, less than about 20%, less than about 15%, less than about 10%, or less than about 5% of the dose that is conventionally administered of dimenthyl fumarate. The conventionally administered dose of dimethyl fumarate is as starting dose 120 mg. After 7 days, the dose is increased to 240 mg (maintenance dose). Accordingly, a sub-therapeutic starting dose of dimethyl fumarate is less than 90 mg, less than 84 mg, less than 72 mg, less than 60 ng, less than 48 mg, less than 36 mg, less than 30 mg, less than 24 mg, less than 18 mg, less than 12 mg or less than 6 mg. A sub-therapeutic maintainance dose of dimethyl fumarate is less than 180 mg, less than 168 mg, less than 144 mg, less than 120 mg, less than 96 mg, less than 72 mg, less than 60 mg, less than 48 mg, less than 36 mg, less than 24 mg or less than 12 mg.

In another embodiment, both, the CD20 ligand and the at least one further active agent are administered in a sub-therapeutic dose, i.e. in a dose that is less than about 75%, less than about 70%, less than about 60%, less than about 50%, less than about 40%, less than about 30%, less than about 25%, less than about 20%, less than about 15%, less than about 10%, or less than about 5% of the dose that is conventionally administered of the CD20 ligand and the at least one further active agent, respectively. For example, the CD20 ligand is administered in a dose that is less than about 75% of the dose that is conventionally administered of the CD20 ligand and the at least one further active agent is administered in a dose that is less than about 75% of the dose that is conventionally administered at least one further active agent. For example, the CD20 ligand is administered in a dose that is less than about 70% of the dose that is conventionally administered of the CD20 ligand and the at least one further active agent is administered in a dose that is less than about 70% of the dose that is conventionally administered at least one further active agent. For example, the CD20 ligand is administered in a dose that is less than about 60% of the dose that is conventionally administered of the CD20 ligand and the at least one further active agent is administered in a dose that is less than about 60% of the dose that is conventionally administered at least one further active agent. For example, the CD20 ligand is administered in a dose that is less than about 50% of the dose that is conventionally administered of the CD20 ligand and the at least one further active agent is administered in a dose that is less than about 50% of the dose that is conventionally administered at least one further active agent. For example, the CD20 ligand is administered in a dose that is less than about 40% of the dose that is conventionally administered of the CD20 ligand and the at least one further active agent is administered in a dose that is less than about 40% of the dose that is conventionally administered at least one further active agent. For example, the CD20 ligand is administered in a dose that is less than about 30% of the dose that is conventionally administered of the CD20 ligand and the at least one further active agent is administered in a dose that is less than about 30% of the dose that is conventionally administered at least one further active agent. For example, the CD20 ligand is administered in a dose that is less than about 25% of the dose that is conventionally administered of the CD20 ligand and the at least one further active agent is administered in a dose that is less than about 25% of the dose that is conventionally administered at least one further active agent. For example, the CD20 ligand is administered in a dose that is less than about 20% of the dose that is conventionally administered of the CD20 ligand and the at least one further active agent is administered in a dose that is less than about 20% of the dose that is conventionally administered at least one further active agent. For example, the CD20 ligand is administered in a dose that is less than about 15% of the dose that is conventionally administered of the CD20 ligand and the at least one further active agent is administered in a dose that is less than about 15% of the dose that is conventionally administered at least one further active agent. For example, the CD20 ligand is administered in a dose that is less than about 10% of the dose that is conventionally administered of the CD20 ligand and the at least one further active agent is administered in a dose that is less than about 10% of the dose that is conventionally administered at least one further active agent. For example, the CD20 ligand is administered in a dose that is less than about 5% of the dose that is conventionally administered of the CD20 ligand and the at least one further active agent is administered in a dose that is less than about 5% of the dose that is conventionally administered at least one further active agent.

In one embodiment MabionCD20DP is administered at a dose of 375 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 280 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 260 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 185 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 150 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 110 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 95 Mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 75 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 55 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 35 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 18 mg/m² body surface and teriflunomide is administered at a dose of 14 mg, less than 10 mg, less than 9 mg, less than 8 mg, less than 7 mg, less than 5 mg, less than 4 mg, less than 3.5 mg, less than 3 mg, less than 2 mg, less than 1.5 mg or less than 0.7 mg.

In one embodiment MabionCD20DP is administered at a dose of 375 mg/m² body surface and fingolimod is administered at a dose of 500 μg, less than 375 μg, less than 350 μg, less than 300 μg, less than 250 μg, less than 200 μg, less than 150 μg, less than 125 μg, less than 100 μg, less than 75 μg, less than 50 μg or less than 25 μg.

In one embodiment MabionCD20DP is administered at a dose of 280 mg/m² body surface and fingolimod is administered at a dose of 500 μg, less than 375 μg, less than 350 μg, less than 300 μg, less than 250 μg, less than 200 μg, less than 150 μg, less than 125 μg, less than 100 μg, less than 75 μg, less than 50 μg or less than 25 μg.

In one embodiment MabionCD20DP is administered at a dose of 260 mg/m² body surface and fingolimod is administered at a dose of 500 μg, less than 375 μg, less than 350 μg, less than 300 μg, less than 250 μg, less than 200 μg, less than 150 μg, less than 125 μg, less than 100 μg, less than 75 μg, less than 50 μg or less than 25 μg.

In one embodiment MabionCD20DP is administered at a dose of 185 mg/m² body surface and fingolimod is administered at a dose of 500 μg, less than 375 μg, less than 350 μg, less than 300 μg, less than 250 μg, less than 200 μg, less than 150 μg, less than 125 μg, less than 100 μg, less than 75 μg, less than 50 μg or less than 25 μg.

In one embodiment MabionCD20DP is administered at a dose of 150 mg/m² body surface and fingolimod is administered at a dose of 500 μg, less than 375 μg, less than 350 μg less than 300 μg, less than 250 μg, less than 200 μg, less than 150 μg, less than 125 μg, less than 100 μg, less than 75 μg, less than 50 μg or less than 25 μg.

In one embodiment MabionCD20DP is administered at a dose of 110 mg/m² body surface and fingolimod is administered at a dose of 500 μg, less than 375 μg, less than 350 μg, less than 300 μg, less than 250 μg, less than 200 μg, less than 150 μg, less than 125 μg, less than 100 μg, less than 75 μg, less than 50 μg or less than 25 μg.

In one embodiment MabionCD20DP is administered at a dose of 95 mg/m² body surface and fingolimod is administered at a dose of 500 μg, less than 375 μg, less than 350 μg, less than 300 μg, less than 250 μg, less than 200 μg, less than 150 μg, less than 125 μg, less than 100 μg, less than 75 μg, less than 50 μg or less than 25 μg.

In one embodiment MabionCD20DP is administered at a dose of 75 mg/m² body surface and fingolimod is administered at a dose of 500 μg, less than 375 μg, less than 350 μg, less than 300 μg, less than 250 μg, less than 200 μg, less than 150 μg, less than 125 μg, less than 100 μg, less than 75 μg, less than 50 μg or less than 25 μg.

In one embodiment MabionCD20DP is administered at a dose of 55 mg/m² body surface and fingolimod is administered at a dose of 500 μg, less than 375 μg, less than 350 μg, less than 300 μg, less than 250 μg, less than 200 μg, less than 150 g, less than 125 μg, less than 100 μg, less than 75 μg, less than 50 μg or less than 25 μg.

In one embodiment MabionCD20DP is administered at a dose of 35 mg/m² body surface and fingolimod is administered at a dose of 500 μg, less than 375 μg, less than 350 μg, less than 300 μg, less than 250 μg, less than 200 mg, less than 150 μg, less than 125 μg, less than 100 μg, less than 75 μg, less than 50 μg or less than 25 μg.

In one embodiment MabionCD20DP is administered at a dose of 18 mg/m² body surface and fingolimod is administered at a dose of 500 μg, less than 375 μg, less than 350 μg, less than 300 μg, less than 250 μg, less than 200 μg, less than 150 mg, less than 125 μg, less than 100 μg, less than 75 μg, less than 50 μg or less than 25 μg.

In one embodiment MabionCD20DP is administered at a dose of 375 mg/m² body surface and dimethyl fumarate is administered at a starting dose of 120 mg and a maintainance dose of 240 mg, a starting dose of less than 90 mg and a maintainance dose of less than 180 mg, a starting dose of less than 84 mg and a maintainance dose of less than 168 mg, a starting dose of less than 72 mg and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mag, a starting dose of less than 24 mg and a maintainance dose of less than 48 mg, a starting dose of less than 18 mg and a maintainance dose of less than 36 mg, a starting dose of less than 12 mg and a maintainance dose of less than 24 mg or a starting dose of less than 6 mg and a maintainance dose of less than 12 mg.

In one embodiment MabionCD20DP is administered at a dose of 280 mg/m² body surface and dimethyl fumarate is administered at a starting dose of 120 mg and a maintainance dose of 240 mg, a starting dose of less than 90 mg and a maintainance dose of less than 180 mg, a starting dose of less than 84 mg and a maintainance dose of less than 168 mg, a starting dose of less than 72 mg and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 mg and a maintainance dose of less than 24 mg or a starting dose of less than 6 mg and a maintainance dose of less than 12 mg.

In one embodiment MabionCD20DP is administered at a dose of 260 mg/m² body surface and dimethyl fumarate is administered at a starting dose of 120 mg and a maintainance dose of 240 mg, a starting dose of less than 90 mg and a maintainance dose of less than 180 mg, a starting dose of less than 84 mg and a maintainance dose of less than 168 mg, a starting dose of less than 72 mg and a maintainance dose of less than 144 mg, a starting dose of less than 60 mg and a maintainance dose of less than 120 mg, a, starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 ma and a maintainance dose of less than 24 mg or a starting dose of less than 6 mg and a maintainance dose of less than 12 mg.

In one embodiment MabionCD20DP is administered at a dose of 185 mg/m² body surface and dimethyl fumarate is administered at a starting dose of 120 mg and a maintainance dose of 240 ng, a starting dose of less than 90 mg and a maintainance dose of less than 180 mg, a starting dose of less than 84 mg and a maintainance dose of less than 168 ng, a starting dose of less than 72 mg and a maintainance dose of less than 144 mg, a starting dose of less than 60 ng and a maintainance dose of less than 120 ng, a starting dose of less than 48 ng and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 mg and a maintainance dose of less than 24 mg or a starting dose of less than 6 mg and a maintainance dose of less than 12 mg.

In one embodiment MabionCD20DP is administered at a dose of 150 mg/m² body surface and dimethyl fumarate is administered at a starting dose of 120 mg and a maintainance dose of 240 ng, a starting dose of less than 90 mg and a maintainance dose of less than 180 mg, a starting dose of less than 84 mg and a maintainance dose of less than 168 mg, a starting dose of less than 72 ng and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 mg and a maintainance dose of less than 24 mg or a starting dose of less than 6 mg and a maintainance dose of less than 12 mg.

In one embodiment MabionCD20DP is administered at a dose of 110 mg/m²n body surface and dimethyl fumarate is administered at a starting dose of 120 mg and a maintainance dose of 240 mg, a starting dose of less than 90 mg and a maintainance dose of less than 180 mg, a starting dose of less than 84 mg and a maintainance dose of less than 168 mg, a starting dose of less than 72 mg and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 mg and a maintainance dose of less than 24 trig or a starting dose of less than 6 mg and a maintainance dose of less than 12 mg.

In one embodiment MabionCD20DP is administered at a dose of 95 mg/m² body surface and dimethyl fumarate is administered at a starting dose of 120 mg and a maintainance dose of 240 mg, a starting dose of less than 90 mg and a maintainance dose of less than 180 mg, a starting dose of less than 84 wig and a maintainance dose of less than 168 mg, a starting dose of less than 72 mg and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 mg and a maintainance dose of less than 24 mg or a starting dose of less than 6 mg and a maintainance dose of less than 12 mg.

In one embodiment MabionCD20DP is administered at a dose of 75 mg/m² body surface and dimethyl fumarate is administered at a starting dose of 120 mg and a maintainance dose of 240 mg, a starting dose of less than 90 mg and a maintainance dose of less than 180 mg, a starting dose of less than 84 mg and a maintainance dose of less than 168 mg, a starting dose of less than 72 mg and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 wig and a maintainance dose of less than 36 mg, a starting dose of less than 12 mg and a maintainance dose of less than 24 mg or a starting dose of less than 6 mg and a maintainance dose of less than 12 mg.

In one embodiment MabionCD20DP is administered at a dose of 55 mg/m² body surface and dimethyl fumarate is administered at a starting dose of 120 wig and a maintainance dose of 240 mg, a starting dose of less than 90 mg and a maintainance dose of less than 180 mg, a starting dose of less than 84 mg and a maintainance dose of less than 168 mg, a starting dose of less than 72 mg and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 mg and a maintainance dose of less than 24 mg or a starting dose of less than 6 mg and a maintainance dose of less than 12 mg.

In one embodiment MabionCD20DP is administered at a dose of 35 mg/m² body surface and dimethyl fumarate is administered at a starting dose of 120 mg and a maintainance dose of 240 mg, a starting dose of less than 90 ng and a maintainance dose of less than 180 mg, a starting dose of less than 84 mg and a maintainance dose of less than 168 mg, a starting dose of less than 72 mg and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 mg and a maintainance dose of less than 24 mg or a starting dose of less than 6 mg and a maintainance dose of less than 12 mg.

In one embodiment MabionCD20DP is administered at a dose of 18 mg/m² body surface and dimethyl fumarate is administered at a starting dose of 120 ng and a maintainance dose of 240 mg, a starting dose of less than 90 mg and a maintainance dose of less than 180 mg, a starting dose of less than 84 mg and a maintainance dose of less than 168 mg, a starting dose of less than 72 mg and a maintainance dose of less than 144 mg, a starting dose of less than 60 mg and a maintainance dose of less than 120 mg, a starting dose of less than 48 mg and a maintainance dose of less than 96 mg, a starting dose of less than 36 mg and a maintainance dose of less than 72 mg, a starting dose of less than 30 mg and a maintainance dose of less than 60 mg, a starting dose of less than 24 mg and a maintainance dose of less than 48 mg, a starting dose of less than 18 mg and a maintainance dose of less than 36 mg, a starting dose of less than 12 mg and a maintainance dose of less than 24 mg or a starting dose of less than 6 mg and a maintainance dose of less than 12 mg.

In a preferred embodiment of the first aspect of the present invention, the CD20 ligand and the at least one further active agent are administered either simultaneously or sequentially or a combination thereof. In one embodiment, the CD20 ligand and the at least one further active agent are administered simultaneously. In another embodiment, the CD20 ligand and the at least one further active agent are administered sequentially.

In a second aspect, the present invention relates to a pharmaceutical composition for use in the treatment of multiple sclerosis comprising the CD20 ligand and the at least one further active agent according to the first aspect of the invention and at least one pharmaceutically acceptable carrier. Preferably, the at least one pharmaceutically acceptable carrier is selected from the group consisting of a diluent, adjuvant, excipient, or vehicle with which the therapeutic agent is administered. More preferably, the at least one pharmaceutically acceptable carrier is an excipient. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffeting agents. The compositions of the invention can take the form of solutions, suspensions, emulsions, tablets, pills, capsules, powders, sustained-release formulations and the like. The composition can be formulated as a suppository, with traditional binders and carriers such as triglycerides.

In a preferred embodiment of the second aspect of the present invention, the at least one further active agent is teriflunomide. In a preferred embodiment of the second aspect of the present invention, the at least one further active agent is fingolimod. In a preferred embodiment of the second aspect of the present invention, the at least one further active agent is dimethyl fumarate.

In a third aspect, the present invention relates to a kit for use in the treatment of multiple sclerosis comprising the CD20 ligand and the at least one further active agent according to first aspect of the invention or a composition according to the second aspect of the invention.

### EXAMPLES

Example 1: Test System and Experimental Materials. The Test System is Described in Table 1 and the Experimental Materials are Listed in Table 2.

TABLE 1
Summary of animal model, housing, randomization and termination

| | |
|---|---|
| Species/Strain: | Mouse/C57BL/6J. |
| Source: | Envigo RMS (Israel), Ltd. |
| Gender: | Female. |
| No. of animals: | n = 174. |
| Age: | Young adults; 18-20 g at study initiation. |
| Body Weight: | Weight variation of animals at the time of treatment initiation should not |
| | exceed ±20% of the mean weight. |
| Animal Health: | The health status of the animals used in this study is examined upon their |
| | arrival. Only animals in good health are acclimatized to laboratory |
| | conditions and are used in the study |
| Acclimation: | At least 5 days |
| Housing: | During acclimation and throughout the entire study duration, animals are |
| | housed within a limited access rodent facility and kept in groups with a |
| | maximum of 10 mice per polypropylene cage. The cages are fitted with |
| | solid bottoms and filled with sterile wood shavings as bedding material |
| Food and | Animals are provided ad libitum with a commercial rodent diet and have |
| Water: | free access to drinking water that is supplied to each cage via |
| | polyethylene bottles with stainless steel sipper tubes. A feed lot analysis |
| | of the diet batch used in the study will be included in the archives with |
| | the study data. Water is monitored periodically |
| Environment: | Automatically controlled environmental conditions are set to maintain |
| | temperature at 17-23° C. with a relative humidity (RH) of 30-70%, a 12:12 |
| | hour light: dark cycle and 15-30 air changes/hour in the study room. |
| | Temperature and RH are monitored daily |
| Identification: | Animals are given a unique animal identification tail mark. This number |
| | also appears on a cage card on the front of each cage. The cage card also |
| | contains the study and group numbers, route of administration, gender, |
| | strain, and all other relevant details as to treatment group |
| Randomization: | During the acclimation period, animals are randomly assigned to |
| | experimental groups according to body weight. Each dosing group is kept |
| | in separate cages to avoid cross-contamination which can occur through |
| | consumption of fecal matter during the study |
| Termination: | At the end of the study, animals are anesthetized with Pentobarbital |
| | Sodium for bleeding and organ collection |
| Justification: | The mouse has been selected as it represented the species of choice for |
| | this experimental animal model. |

TABLE 2
Experimental materials

| Materials | Name |
|---|---|
| Vehicle | Saline |
| Test Items | MabionCD20DP |
| | Teriflunomide |
| | Fingolimod |
| | Dimethyl Fumarate |
| Positive control | Dexamethasone |
| Sensitizing Items | MOG 35-55 Rodent |
| | PBS |
| | Complete Freund's Adjuvant (CFA) |
| | Pertussis Toxin (PT) |
| Euthanasia items | Pentobarbital Sodium |

TABLE 3
Summary of test materials preparation

| | | Concentration of | Group |
|---|---|---|---|
| Tested material | Vehicle | tested material | no. |
| Dexamethasone | Ethanol | 1 mg/kg; | 4 |
| | | 0.1 mg/mL | |
| Teriflunomide | 0.5% Methylcellulose | 2.87 mg/kg; | 6 |
| therapeutic | containing 0.5% | 0.574 mg/mL | |
| Teriflunomide sub- | Tweent ® 80 | 0.957 mg/kg; | 5, 7 |
| therapeutic | | 0.19 mg/mL | |
| Fingolimod | distilled water | 0.1 mg/kg, | 9 |
| therapeutic | | 0.02 mg/mL | |
| Fingolimod sub- | | 0.034 mg/kg, | 8, 10 |
| therapeutic | | 0.0068 mg/mL | |
| Dimethyl fumarate | 0.5% Methylcellulose | 49 mg/kg, | 11 |
| therapeutic | containing 0.5% | 9.8 mg/mL | |
| Dimethyl fumarate | Tween ® 80 | 16 mg/kg, | 12 |
| sub-therapeutic | | 3.2 mg/mL | |
| MabionCD20DP | Saline | 5 mg/kg, | 14 |
| therapeutic | | 1 mg/mL | |
| MabionCD20DP | Saline | 0.5 mg/kg, | 7, 10, |
| sub-therapeutic | | 0.1 mg/mL | 12, 13 |

TABLE 4
Summary of experimental groups

| | | | | Dose | Volume | |
|---|---|---|---|---|---|---|
| Group | Group | Group/Test | | Level | Dosage | Regime |
| Number | Size | Material | Route | (mg/kg) | (ml/kg) | respectively |
| 1 | N = 5 | Naive | N/A | N/A | N/A | N/A |
| 2 | N = 12 | EAE disease only | N/A | | | N/A |
| 3 | N = 12 | Vehicle control | IV¹ | | | 0-2 days, OD² |
| 4 | N = 12 | Dexamethasone | PO³ | 1 | 10 | 0-35 days, OD |
| 5 | N = 12 | Teriflunornide | PO | 0.957 | 5 | |
| 6 | N = 12 | Teriflunornide | | 2.87 | 5 | |
| 7 | N = 12 | MabionCD20DP + Teriflunomide | IV/PO | 0.5 + 0.957 | 5 | 0-2 days, OD; |
| | | | | | | 0-35 days, OD |
| 8 | N = 12 | Fingolimod | PO | 0.034 | 5 | 0-35 days, OD |
| 9 | N = 12 | Fingolimod | | 0.1 | 5 | |
| 10 | N = 12 | MabionCD20DP + Fingolimod | IV/PO | 0.5 + 0.034 | 5 | 0-2 days, OD; |
| | | | | | | 0-35 days, OD |
| 11 | N = 12 | Dimethyl fumarate | PO | 49 | 5 | 0-35 days, BD⁴ |
| 12 | N = 12 | MabionCD20DP + | IV/PO | 0.5 + 16 | 5 | 0-2 days, OD; |
| | | Dimethyl fumarate | | | | 0-35 days, BD |
| 13 | N = 12 | MabionCD20DP | IV | 0.5 | 5 | 0-2 days, OD |
| 14 | N = 12 | MabionCD20DP | IV | 5 | 5 | |

1 - IV - intravenous administration
2 - OD - once daily
3 - PO - pre os (oral administration)
4 - BD - bidaily

### Example 2: Characterization of MabionCD20DP

MabionCD20DP is a recombinant, chimeric murine/human monoclonal antibody representing a gly cosylated immunoglobulin with human IgG1 constant regions and murine light-chain and heavy-chain variable region sequences. MabionCD20DP is a clear, colorless liquid without the presence of solid particulate in the form of needles, threads, lint, concentrate for solution formulated for IV administration as a sterile product. The pH is adjusted to 6.5.

TABLE 5
DP composition

| | | | Reference to |
|---|---|---|---|
| Component | Function | Quantity/vial | Standards |
| MabionCD20DP | Active | 10 mg/mL | — |
| | ingredient | (10 or 50 mL vial) | |
| Polysorbate 80 | Excipient | 0.7 mg/mL | Ph.Eur |
| Sodium citrate | Tonicifier/ | 7.35 mg/mL | Ph.Eur |
| | stabilizer | | |
| Sodium chloride | Tonicifier/ | 9 mg/mL | Ph.Eur |
| | stabilizer | | |
| Water | Diluent | — | Ph.Eur |

### Example 3: EAE Mouse Model

Experimental allergic encephalomyelitis (EAE) is a central nervous system (CNS) autoimmune demyelinating disease that mimics many of the clinical and pathologic features of multiple sclerosis (MS). The proteolipid protein (PLP) murine model consists of a sensitization period, induced by the single subcutaneous (SC) injection of PLP in complete Freund's adjuvant (CFA), spread out over 2 sites on the shaven back, on Study Day 0, followed by IP supplemental immunostimulation with pertussis toxin (PT) carried out once at the day of EAE induction—study day 0 and once again 48 hours later—on study day 2.

Throughout the 36-day study, general clinical examinations were performed and recorded if any unexpected abnormalities are observed.

Body weight loss can be the first sign of disease initiation, while a sudden marked weight gain tends to accompany remission of EAE symptoms. Therefore, determination of individual body weights of animals was made shortly before EAE induction on Study Day 0 and was monitored on a daily basis throughout the 35-day observation period.

All animals were examined for signs of any neurological responses and symptoms prior to EAE induction (Study Day 0) and thereafter, examined on a daily basis throughout the entire 35-day observation period. EAE reactions are scored and recorded according to a 0-15 scale. The clinical score is determined by summing the score from each section (Weaver at al, 2005):

| Signs/Symptoms | | Grade |
|---|---|---|
| Tail | No Signs | 0 |
| | Half paralyzed tail | 1 |
| | Fully paralyzed tail | 2 |
| For each of the | No Signs | 0 |
| hind or forelimbs | Weak or altered gait | 1 |
| | Paresis | 2 |
| | Fully paralyzed limb | 3 |
| Mortality | — | 15 |

Animals were anesthetized with a combination of ketamine 35 mg/kg-IP and xylazine HCl 8 mg/kg-IP. Under anesthesia, the animals were bled via cardiac puncture and the plasmas were prepared and stored at −80° C.

### Example 4: Combination of MabionCD20DP with Teriflunomide

The aim of the study was to evaluate the effect of MabionCD20DP in the MOG-induced experimental allergic encephalomyelitis (EAE) murine model.

Experimental materials: Saline—Vehicle and Negative control, MOG 35-55 Rodent—Sensitizing items, PBS—Sensitizing items, Complete Freund's Adjuvant (CFA)—Sensitizing items, Pertussis Toxin (PT)—Sensitizing items, Dexamethasone—Positive control, MabionCD20DP—Test item, Teriflunomide—Test item, Pentobarbital Sodium—Euthanasia items.

Experimental groups: during the acclimation period, animals are randomly assigned to 15 experimental groups according to body weight. Each dosing group was kept in separate cages to avoid cross-contamination which can occur through consumption of fecal matter during the study. Animals are given a unique animal identification tail mark. The groups were: Group 1—Naïve; 5 animals, Group 2—EAE disease only; 12 animals, Group 3—Vehicle Control; 12 animals, Group 4—Dexamethasone; 12 animals, Group 5—Teriflunomide; 0.957 mg/kg; administration once daily; Days 0-35; 12 animals, Group 6—Teriflunomide; 2.87 mg/kg; administration once daily; Days 0-35; 12 animals, Group 7—Mabion CD20 and Teriflunomide; 0.5 mg/kg and 0.957 mg/kg respectively; MabionCD20DP-Days 0-2 and Teriflunomide—administration once daily; Days 0-35; 12 animals, Group 13-MabionCD20DP; 0.5 mg/kg; administration on days 0-2; 12 animals, Group 14-MabionCD20DP; 5 mg/kg; administration on days 0-2; 12 animals. At the end of the study, animals are anesthetized with Pentobarbital Sodium for bleeding and organ collection.

Preparation of experimental materials: MabionCD20DP—group 14, MabionCD20DP preparation at a dose level of 5 mg/kg, at a concentration of 1 mg/ml, and at a dose volume of 5 ml/kg proceeded as follows: 4 mg of MabionCD20DP (10 mg/ml) was taken and resolved in 4 ml of saline provided as a ready to use. MabionCD20DP—group 7: MabionCD20DP preparation at a dose level of 0.5 mg/kg, at a concentration of 0.1 mg/mil, and at a dose volume of 5 ml/kg proceeded as follows: 1 ml of MabionCD20DP at a concentration of 1 mg/ml was taken and resolved in 9 ml of saline provided as a ready to use. Teriflunomide—group 6: Preparation of Teriflunomide at a dose level of 2.87 mg/kg, at a concentration of 0.574 mg/ml, and at a dose volume of 5 ml/kg proceeded as follows: 3.44 mg of teriflunomide was resolved in 6 ml of 0.5% Methylcellulose/0.5% Tween. Teriflunomide—groups 5 and 7: Preparation of Teriflunomide at a dose level of 0.957 mg/kg, at a concentration of 0.19 mg/ml, and at a dose volume of 5 mil/kg proceeded as follows: 2.28 mg of teriflunomide was resolved in 12 ml of 0.5% Methylcellulose/0.5% Tween.

Throughout the 36-day study, the animals were examined as specified in Example 3.

Blood Collection: Animals were anesthetized with a combination of ketamine 35 mg/kg and xylazine HCl 8 mg/kg IP. Under anesthesia, the animals were bled via cardiac puncture. The plasma were prepared as follows: whole blood samples were collected in Eppendorf tubes with EDTA containing the anticoagulant (K₃ EDTA), the samples were centrifuged in a microfuge for 5 minutes at 2500 rpm, the plasma were collected and stored at −80° C. Statistical data evaluation: statistical evaluation was primarily based on the relative recorded changes in neurological symptoms, body weights and clinical score expressed as mean group values obtained in all treated groups vs. those of the Vehicle Control. Where appropriate, analysis of the data was applied to determine significance of treatment effects.

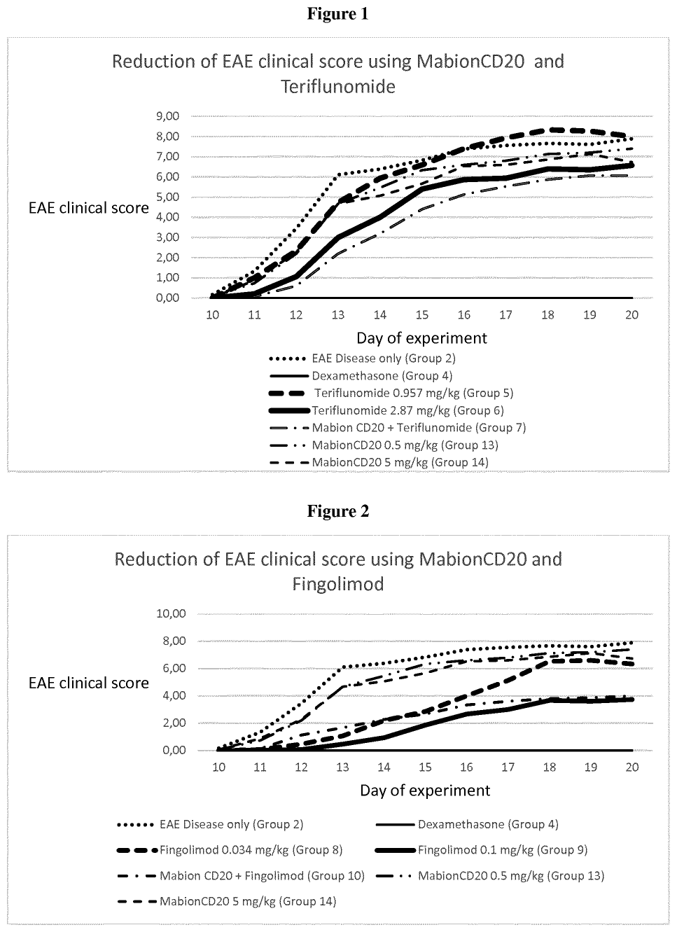

The results of this experiment are reproduced in FIG. 1. While it can be seen that teriflunomide in therapeutic dose shows a reduction of the disease parameters, teriflunomide in subtherapeutic dose alone has no therapeutic effect. If, however, teriflunomide is administered in subtherapeutic dose in combination with MabionCD20DP a reduction of the disease comparable to teriflunomide in therapeutic dose can be observed. Thus, the combination of a subtherapeutic dose of teriflunomide and MabionCD20DP increases efficacy of the treatment and has a comparable therapeutic effect as teriflunomide administered alone in therapeutic dose.

### Example 5: Combination of MabionCD20DP with Dimethyl Fumarate

The aim of the study was to evaluate the effect of MabionCD20DP in the MOG-induced experimental allergic encephalomyelitis (EAE) murine model.

Experimental materials: Saline Vehicle and Negative control, MOG 35-55 Rodent—Sensitizing items, PBS—Sensitizing items, Complete Freund's Adjuvant (CFA)—Sensitizing items, Pertussis Toxin (PT)—Sensitizing items, Dexamethasone—Positive control, MabionCD20DP—Test item, Dimethyl fumarate—Test item, Pentobarbital Sodium—Euthanasia items.

Experimental groups: during the acclimation period, animals are randomly assigned to 15 experimental groups according to body weight. Each dosing group was kept in separate cages to avoid cross-contamination which can occur through consumption of fecal matter during the study. Animals are given a unique animal identification tail mark. The groups were: Group 1—Naïve: 5 animals, Group 2—EAE disease only; 12 animals, Group 3—Vehicle Control; 12 animals, Group 4—Dexamethasone; 12 animals, Group 11—Tecfidera—Dimethyl Fumarate; 49 mg/kg; administration bi-daily; Days 0-35; 12 animals, Group 12-MabionCD20DP and Tecfidera; 0.5 mg/kg and 16 mg/kg respectively; MabionCD20DP—administration on days 0-2 and Tecfidera—administration bi-daily; Days 0-35; 12 animals, Group 13—MabionCD20DP; 0.5 mg/kg; administration on days 0-2; 12 animals, Group 14-MabionCD20DP; 5 mg/kg; administration on days 0-2; 12 animals. At the end of the study, animals are anesthetized with Pentobarbital Sodium for bleeding and organ collection.

Preparation of experimental materials: MabionCD20DP—group 14, MabionCD20DP preparation at a dose level of 5 mg/kg, at a concentration of 1 mg/ml, and at a dose volume of 5 ml/kg proceeded as follows: 4 mg of MabionCD20DP (10 mg/ml) was taken and resolved in 4 ml of saline provided as a ready to use. MabionCD20DP—group 12: MabionCD20DP preparation at a dose level of 0.5 mg/kg, at a concentration of 0.1 mg/ml, and at a dose volume of 5 ml/kg proceeded as follows: 1 ml of MabionCD20DP at a concentration of 1 mg/ml was taken and resolved in 9 ml of saline provided as a ready to use. Teriflunomide—group 6: Preparation of Teriflunomide at a dose level of 2.87 mg/kg, at a concentration of 0.574 mg/ml, and at a dose volume of 5 ml/kg proceeded as follows: 3.44 mg of teriflunomide was resolve in 6 ml of 0.5% Methylcellulose/0.5% Tween. Dimethyl fumarate—group 11—dose level of 49 mg/kg, at a concentration of 9.8 mg/ml, and at a dose volume of 5 ml/kg proceeded as follows: 137.2 mg of dimethyl fumarate was resolved in 14 ml of 0.5% Methylcellulose/0.5% Tween. Group 12—dose level of 16 mg/kg, at a concentration of 3.2 mg/ml, and at a dose volume of 5 ml/kg proceeded as follows: 89.6 mg of Tecfidera was resolved in 28 ml of 0.5% Methylcellulose/0.5% Tween.

Throughout the 36-day study, the animals were examined as specified in Example 3.

Blood Collection: Animals were anesthetized with a combination of ketamine 35 mg/kg and xylazine HCl 8 mg/kg IP. Under anesthesia, the animals were bled via cardiac puncture. The plasma were prepared as follows: whole blood samples were collected in Eppendorf tubes with EDTA containing the anticoagulant (K₃ EDTA), the samples were centrifuged in a microfuge for 5 minutes at 2500 rpm, the plasma were collected and stored at −80° C.

Statistical data evaluation: statistical evaluation was primarily based on the relative recorded changes in neurological symptoms, body weights and clinical score expressed as mean group values obtained in all treated groups vs. those of the Vehicle Control. Where appropriate, analysis of the data was applied to determine significance of treatment effects.

The results of this experiment are reproduced in FIG. 2. It can be seen that dimethyl fumarate in therapeutic dose shows a reduction of the disease parameters. If dimethyl furanoate is administered in subtherapeutic dose in combination with MabionCD20DP a reduction of the disease to an extent comparable to the therapeutic dose of dimethyl fumarate can be observed. Thus, the combination of a subtherapeutic dose of dimethyl fumarate and MabionCD20DP has a comparable therapeutic effect as dimethyl fumarate administered alone in therapeutic dose.

### Example 6: Combination of MabionCD20DP with Fingolimod

The aim of the study was to evaluate the effect of MabionCD20DP in the MOG-induced experimental allergic encephalomyelitis (EAE) murine model.

Experimental materials: Saline—Vehicle and Negative control. MOG 35-55 Rodent—Sensitizing items, PBS—Sensitizing items, Complete Freund's Adjuvant (CFA)—Sensitizing items, Pertussis Toxin (PT)—Sensitizing items, Dexamethasone—Positive control, MabionCD20DP—Test item, Teriflunomide—Test item, Pentobarbital Sodium—Euthanasia items.

Experimental groups: during the acclimation period, animals are randomly assigned to 15 experimental groups according to body weight. Each dosing group was kept in separate cages to avoid cross-contamination which can occur through consumption of fecal matter during the study. Animals are given a unique animal identification tail mark. This number also appears on a cage card on the front of each cage. The cage card also contains the study and group numbers, route of administration, gender and strain. The groups were: Group 1—Naïve; 5 animals, Group 2—EAE disease only; 12 animals, Group 3—Vehicle Control; 12 animals, Group 4—Dexamethasone; 12 animals, Group 8—Fingolimod; 0.034 mg/kg; administration once daily; Days 0-35; 12 animals, Group 9—Fingolimod; 0.1 mg/kg; administration once daily; Days 0-35: 12 animals, Group 10—MabionCD20DP and Fingolimod; 0.5 mg/kg and 0.034 mg/kg respectively; MabionCD20DP—administration on days 0-2 and Fingolimod—administration once daily; Days 0-35; 12 animals, Group 13—MabionCD20DP; 0.5 mg/kg; administration on days 0-2; 12 animals, Group 14—MabionCD20DP; 5 mg/kg; administration on days 0-2; 12 animals. At the end of the study, animals are anesthetized with Pentobarbital Sodium for bleeding and organ collection.

Preparation of experimental materials: MabionCD20DP—group 14, MabionCD20DP preparation at a dose level of 5 mg/kg, at a concentration of 1 mg/ml, and at a dose volume of 5 ml/kg proceeded as follows: 4 mg of MabionCD20DP (10 mg/ml) was taken and resolved in 4 ml of saline provided as a ready to use. MabionCD20DP—group 7: MabionCD20DP preparation at a dose level of 0.5 mg/kg, at a concentration of 0.1 mg/ml, and at a dose volume of 5 mil/kg proceeded as follows: 1 ml of MabionCD20DP at a concentration of 1 mg/ml was taken and resolved in 9 ml of saline provided as a ready to use. Fingolimod—Group 9—dose level of 0.1 mg/kg, at a concentration of 0.02 mg/ml, and at a dose volume of 5 ml/kg proceeded as follows: 2 mg of Fingolimod was dissolve in 10 ml of distilled water to obtain a stock solution of 0.2 mg/ml (stored at −20° C. as 0.4 ml aliquots). Before each dosing, the aliquot was thaw and diluted with 3.6 ml of distilled water to obtain a working solution at a concentration of 0.02 mg/ml. Fingolimod—groups 8 and 10—dose level of 0.034 mg/kg, at a concentration of 0.0068 mg/mil, and at a dose volume of 5 ml/kg proceeded as follows: 1.5 ml of Fingolimod stock solution at a concentration of 0.02 mg/ml was taken and diluted with 3 ml of distilled water to obtain a working solution at a concentration of 0.0068 mg/mil.

Throughout the 36-day study, the animals were examined as specified in Example 3.

Blood Collection: Animals were anesthetized with a combination of ketamine 35 mg/kg and xylazine HCl 8 mg/kg IP. Under anesthesia, the animals were bled via cardiac puncture. The plasma were prepared as follows: whole blood samples were collected in Eppendorf tubes with EDTA containing the anticoagulant (K₃ EDTA) the samples were centrifuged in a microfuge for 5 minutes at 2500 rpm, the plasma were collected and stored at −80° C. Statistical data evaluation: statistical evaluation was primarily based on the relative recorded changes in neurological symptoms, body weights and clinical score expressed as mean group values obtained in all treated groups vs. those of the Vehicle Control, Where appropriate, analysis of the data was applied to determine significance of treatment effects.

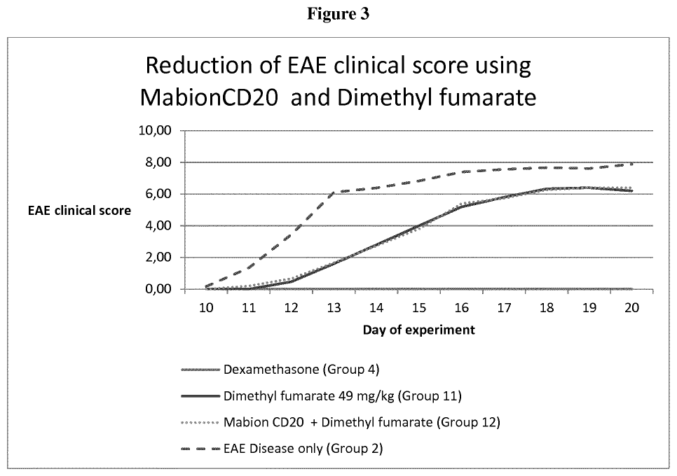

The results of this experiment are reproduced in FIG. 3. While it can be seen that fingolimod in therapeutic dose shows a reduction of the disease parameters, fingolimod in subtherapeutic dose alone has only limited therapeutic effect. If, however, fingolimod is administered in subtherapeutic dose in combination with MabionCD20DP a reduction of the disease to an extent between subtherapeutic dose of fingolimod and therapeutic dose of fingolimod can be observed. Thus, the combination of a subtherapeutic dose of fingolimod and MabionCD20DP increases efficacy of the treatment.

1. A method of treating multiple sclerosis, comprising administration of a CD20 ligand and at least one further active agent, wherein the at least one further active agent decreases lymphocyte count.

2. The method according to claim 1, wherein the CD20 ligand is an antigen binding protein, an antibody or fragment thereof.

3. The method according to claim 2, wherein the CD20 ligand is an antibody or fragment thereof.

4. The method CD20 ligand and the at least one further active agent for use according to any of the preceding claims of claim 1, wherein the CD20 ligand:
a. comprises a combination of a light chain variable domain and a heavy chain variable domain selected from the group of combinations consisting of:
a light chain variable domain having a sequence with at least 90% identity to SEQ ID NO: 1 and
a heavy chain variable domain having a sequence with at least 90% identity to SEQ ID NO: 2;
b. comprises a complementary determining region 3 of the heavy chain (CDRH3) comprising or consisting of the amino acid sequence of SEQ ID NO: 7 and a complementary determining region 3 of the light chain (CDRL3) comprising or consisting of the amino acid sequence of SEQ ID NO: 8; and/or
c. competes for binding to CD20 with at least one antibody selected from the group consisting of rituximab, ibritumomab, obinutuzumab, ofatumumab, tositumomab and ocrelizumab.

5. The method according to claim 4b, wherein the CD20 ligand further comprises one or more selected from the group consisting of a CDRH1 comprising or consisting of the amino acid sequence of SEQ ID NO: 3, a CDRH2 comprising or consisting of the amino acid sequence of SEQ ID NO: 5, a CDRL1 comprising or consisting of the amino acid sequence of SEQ ID NO: 4 and a CDRL2 comprising or consisting of the amino acid sequence of SEQ ID NO: 6.

6. The method CD20 ligand and the at least one further active agent for use according to any of the preceding claims of claim 1, wherein the active agent is a small molecule drug.

7. The method CD20 ligand and the at least one further active agent for use according to any of the preceding claims of claim 1, wherein the active agent is:
(i) an inhibitor of activated T-cells and/or B-cells;
(ii) an antagonist of transcription factor NF-□B;
(iii) an antagonist of sphingosine-1-phosphate (S1P) receptor(s), preferably selected from the group consisting of fingolimod, ponesimod (ACT128800), siponimod (BAF312), ozanimod (RPC1063), ceralifimod (ONO-4641), GSK2018682, and MT-1303;
(iv) an activator of the nuclear factor (erythroid-derived 2)-like 2 (Nrf2) pathway; and/or
(v) an inhibitor of dihydroorotate dehydrogenase, preferably selected from the group leflunomide and teriflunomide.

8. The method of claim 1, wherein the active agent is a compound selected from the group consisting of 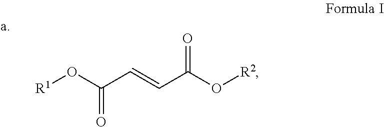
wherein
R1 and R2 are the same or different and are independently selected from the group consisting of linear, branched or cyclic, saturated or unsaturated C1-20alkyl which may be optionally substituted with halogen (Cl, F, I, Br), hydroxy, C1-4alkoxy, nitro or cyano; preferably wherein R1 and R2 are independently selected from the group consisting of C1-5alkyl, such as methyl, ethyl, n-propyl, isopropyl, n-butyl, sec-butyl, t-butyl, pentyl, cyclopentyl; more preferably wherein R1 and R2 are the same;
or a pharmaceutically acceptable form thereof; 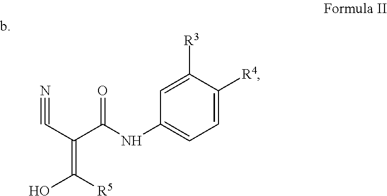
R3 and R4 are the same or different and are independently selected from the group consisting of carboxyl, halogen, hydrogen, trihalomethyl and NO2, substituted or unsubstituted aryl; and
R5 is selected from the group consisting of aryl, alkyl, alkenyl and alkynyl;
or a pharmaceutically acceptable form thereof; 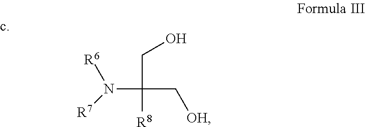
wherein
R6 and R7 are the same or different and are independently selected from the group consisting of hydrogen, alkyl, or acyl;
R8 is a phenylakyl wherein alkyl is a straight- or branched C6-20 carbon chain; or
a phenylalkyl wherein alkyl is a straight- or branched C1-30 carbon chain wherein said phenlyalkyl is substituted by a straight- or branched C6-20 carbon chain optionally substituted by halogen, a straight- or branched C6-20 alkoxy chain optionally substituted by halogen, a straight- or branched C6-20 alkenyloxy, phenyl-C1-14 alkoxy, halophenyl-C1-4 alkoxy, phenyl-C1-14 alkoxy-C1-14 alkyl, phenoxy-C1-4 alkoxy or phenoxy-C1-4alkyl, cycloalkylalkyl substituted by C6-20 alkyl, heteroarylalkyl substituted by C6-20 alkyl, heterocyclic C6-20 alkyl or heterocyclic alkyl substituted by C2-20alkyl; preferably wherein R8 is optionally substituted 2-(4-octylphenyl)ethyl;
or a pharmaceutically acceptable form thereof;

9. The method of claim 1, wherein the at least one further active agent is selected from the group consisting of dimethyl fumarate, leflunomide, teriflunomide and fingolimod or a pharmaceutically acceptable form thereof.

10. The method of claim 1, wherein multiple sclerosis is selected from the group consisting of clinically isolated syndrome (CIS), relapsing-remitting multiple sclerosis (RRMS), primary progressive multiple sclerosis (PPMS), progressive-relapsing multiple sclerosis (PRMS) and secondary progressive multiple sclerosis (SPMS).

11. The method of claim 1, wherein the CD20 ligand and/or the at least one further active agent are administered in a sub-therapeutic dose.

12. The method of claim 1, wherein the CD20 ligand and the at least one further active agent are administered either simultaneously or sequentially or a combination thereof.

13. A pharmaceutical composition for use in the treatment of multiple sclerosis comprising the CD20 ligand and the at least one further active agent according claim 1 and at least one pharmaceutically acceptable carrier.

14. The pharmaceutical composition for use according to claim 13, wherein the at least one further active agent is teriflunomide or fingolimod.

15. A kit for use in the treatment of multiple sclerosis comprising the CD20 ligand and the at least one further active agent according claim 1.

16. A kit for use in the treatment of multiple sclerosis comprising the pharmaceutical composition of claim 13.
